(19) United States
(12) Patent Application Publication
(10) Pub. No.: US 2002/0156112 A1
(43) Pub. Date: Oct. 24, 2002

(54) Endostatin-like angiogenesis inhibition

(76) Inventors: R. Shoshana Bamdad, NY, NY (US); Cynthia C. Bamdad, (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/003,681

(22) Filed: Nov. 15, 2001

Related U.S. Application Data
(60) Provisional application No. 60/248,865, filed on Nov. 15, 2000.
(60) Provisional application No. 60/277,922, filed on Mar. 22, 2001.

(51) Int. Cl. A61K 31/4245; A61K 31/4174; A61K 31/4015; A61K 31/365; A61K 31/255

(52) U.S. Cl. 514/400; 514/424; 514/364; 514/553

(57) ABSTRACT

A treatment for cancer is provided. The treatment may include administering a therapeutic amount of L-histidine, D-cycloserine, quisqualic acid or suramin or analogs thereof.

### RELATED APPLICATIONS

This application claims the benefit of priority to U.S. provisional patent application serial No. 60/248,865, filed Nov. 15, 2000, and No. 60/277,922, filed Mar. 22, 2001.

### FIELD OF THE INVENTION

The invention relates to treatments for cancer, and particularly to treatments using angiogenesis inhibitors.

### BACKGROUND OF THE INVENTION

Angiogenesis is the name given to the in vivo process of new blood vessel formation. It is widely believed that cancer may be effectively treated by reducing or eliminating the supply of blood to a tumor. Angiogenesis inhibitors are a class of compounds that somehow act to interrupt the process of new blood vessel formation. Because adults do not, in general, require much new blood vessel formation, it is thought that angiogenesis inhibitors can be effective treatments against cancer, while having a minimum of negative side effects.

The first angiogenesis inhibitors that were identified, angiostatin and endostatin, are native proteins that were first noticed because they appeared at greater concentration in the urine of animals with tumors than those without tumors. Evidence has been presented that showed that the administration of these proteins to animals with cancerous tumors, resulted in the inhibition of the growth of the tumors, presumably by choking off their blood supply. A drawback of using angiostatin and endostatin as cancer therapeutics is that they are proteins, which are hard to administer, easily degraded by the body and extremely expensive to produce. In fact it is questionable whether enough of these protein therapeutics could be produced to treat all the required cancer patients. For these reasons, it would be advantageous to have a rapid method for identifying new compounds, including synthetic compounds, that act to inhibit angiogenesis.

For several reasons, it has been difficult to identify new angiogenesis inhibitors. First, the biological process of vascularization is not yet well understood. Therefore, the biological targets of the few known angiogenesis inhibitors are either not yet identified or the validity proposed molecular targets is in question. Secondly, the few assays that are used to identify new angiogenesis inhibitors are for the most part functional, cell based assays. These functional assays include the Matrigel Assay (Nicosia, R. F. and A. Ottinetti, In Vitro Cell. Dev. Biol. (1990), Vol. 26:119; and Kubota, Y. Kleinman, H. K., Martin, G. R. and Lawley, T. J. (1988), J. Cell. Biol. Vol. 107, 1589-1598), the chick chorioallantoic membrane assay (Ribatti, D., Vacca, A., Roncali, L. and Danmacco, F., Int. J. Dev. Biol (1996)), the rat aortic ring assay ((Nicosia, R. F. and A. Ottinetti (1990), Lab. Invest. 63, 115-122), and the collagen migration assay (Schor, S. C., Allen, T. D., and Harrison, C. J. (1980) J. Cell. Sci. Vol 46, 171-186). These assays are expensive, time-consuming and not compatible with high throughput.

The present invention solves these problems by providing a molecular target of the known angiogenesis inhibitor, endostatin and by providing a high throughput, nanoparticle-based in vitro assay that rapidly identifies compounds, both natural and synthetic that inhibit angiogenesis by mimicking the effect of endostatin. By identifying synthetic compounds that act to inhibit angiogenesis, the invention provides yet another advantage; synthetic compounds are readily modified and optimized to produce analogs that are more effective than the parent compound. The complex tertiary structure of proteins and antibodies makes it difficult or impossible to optimize them.

### SUMMARY OF THE INVENTION

The present invention involves, in one aspect, methods for treating patients susceptible or exhibiting symptoms of cancer, and in particular, metastatic tumors. The methods may involve, for example, the administration of synthetic replacements, or mimics, of endostatin.

In one aspect, a treatment method comprises treating a human patient susceptible to or exhibiting symptoms of invasive cancer, by administering to the patient a therapeutically effective amount of a composition. The composition may be, for example, L-histidine, quisqualic acid, D-cycloserine, suramin or analogs of any of these. The administering of the therapeutically effective amount of the composition may not be otherwise indicated for the patient.

In another aspect, a treatment method comprises treating a human patient susceptible to or exhibiting symptoms of metastatic tumors, by administering to the patient a therapeutically effective amount of a composition. The composition may be, for example, L-histidine, quisqualic acid, D-cycloserine, suramin or analogs of any of these. The administering of the therapeutically effective amount of the composition may not be otherwise indicated for the patient.

In another aspect, a treatment method comprises treating a human patient where angiogenesis inhibition is indicated, by administering to the patient a therapeutically effective amount of a composition. The composition may be, for example, L-histidine, quisqualic acid, D-cycloserine, suramin or analogs of any of these. The administering of the therapeutically effective amount of the composition may not be otherwise indicated for the patient.

In another aspect, a treatment method comprises treating a human patient wherein treatment with endostatin has been indicated, by administering to the patient a therapeutically effective amount of a composition. The composition may be, for example, L-histidine, quisqualic acid, D-cycloserine, suramin or analogs of any of these.

Other advantages, novel features, and objects of the invention will become apparent from the following detailed description of the invention when considered in conjunction with the accompanying drawings, which are schematic and which are not intended to be drawn to scale. In the figures, each identical or nearly identical component that is illustrated in various figures is represented by a single numeral. For purposes of clarity, not every component is labeled in every figure, nor is every component of each embodiment of the invention shown where illustration is not necessary to allow those of ordinary skill in the art to understand the invention.

### BRIEF DESCRIPTION OF THE FIGURES

FIG. 1 provides the chemical structure of suramin.

FIG. 2 provides the chemical structure of histidine.

FIG. 3 provides the chemical structure of D-cycloserine.

FIG. 4 provides the chemical structure of (+)-quisqualic acid.

FIG. 5 is a photocopy of a digital photograph of a colorimetric nanoparticle experiment in which gold colloids that were derivatized with biotin-SAMs turned blue with the addition of streptavidin, its binding partner, while colloids bearing SAMs that did not present biotin did not turn color and remained pink.

FIG. 6 is a photocopy of a digital photo of a calorimetric nanoparticle experiment that shows that the GRGDS-containing peptide (HHHHHHSSSSGSSSSGSSSSGGRGDSGRGDS) interacts with dimeric endostatin (wells A1 and 2) and that this interaction is competitively inhibited by the addition of full-length vitronectin (well B1), which shows that the peptide substitutes for vitronectin in binding to endostatin.

FIG. 7 is a photocopy of a digital photo of a drug screening plate in which drug candidates were separately tested in wells of a multi-well plate for their ability to interrupt the endostatin-GRGDS-motif peptide interaction. The pink color of well C9 indicates that it contains a drug that mimics endostatin.

### DETAILED DESCRIPTION OF THE INVENTION

International patent application serial number PCT/US00/01997, filed Jan. 25, 2000 by Bamdad et al., entitled “Rapid and Sensitive Detection of Aberrant Protein Aggregation in Neurodegenerative Diseases” (published as WO 00/43791 on Jul. 27, 2000), International patent application serial number PCT/US00/01504, filed Jan. 21, 2000 by Bamdad, et al, entitled “Interaction of Colloid-Immobilized Species with Species on Non-Colloidal Structures” published as WO 00/34783 on Jul. 27, 2000), commonly-owned, copending U.S. patent application Ser. No. 09/602,778, filed Jun. 23, 2000 by Bamdad et al., entitled “Interaction of Colloid-Immobilized Species with Species on Non-Colloidal Structures”; and commonly-owned, copending U.S. patent application Ser. No. 09/631,818, filed Aug. 3, 2000 by Bamdad et al., entitled “Rapid and Sensitive Detection of Protein Aggregation” all are incorporated herein by reference.

There is a need to produce and/or identify new angiogenesis inhibitors, because most of the existing angiogenesis inhibitors are proteins. This means that unlike synthetic drugs, they are difficult to administer, expensive to produce and easily degraded by the body. Additionally, because these proteins are native and present in healthy persons, albeit at much lower levels, there is a concern that administering them in high doses may interfere with some of the normal biological functions. It would therefore be an advantage to have available a system that rapidly identified new angiogenesis inhibitors using a technique compatible with high throughput.

Colloidal assays may provide effective and efficient techniques for screening a wide variety of endostatin mimics. Some of these techniques are described in detail in U.S. patent application Ser. No. 09/631,818, Bamdad and Bamdad, which is hereby incorporated by reference herein, as well as International Patent Publication nos. WO 00/43791 and WO 00/34783, referenced above.

One approach to developing a high throughput drug screen that identifies angiogenesis inhibitors is to identify a relevant biological interaction that promotes angiogenesis, and screen for compounds that disrupt it. The cell surface receptor, αVβ3, has been implicated in promoting metastasis and angiogenesis (Li, X., Regezi, J., Ross, F. P., Blystone, S., Llic, D., Leong, S. P., and Ramos, D. M., Integrin αVβ3 mediates K1735 murine melanoma cell motility in vivo and in vitro, (2001) J. Cell. Sci. Vol. 114 (14): 2665-2672). It is thought that this receptor mediates angiogenesis through an interaction with a cell adhesion molecule, vitronectin (Hynes, R. O. (1987) Cell, Vol. 48, 549-554). Specifically, it is the GRGDS motif derived from vitronectin that the αVβ3 receptor binds to (Standker, L., Enger, A., Schalz-Knappe, P., Wohn, K., Matthias, G., Raida, M., Forssmann, W., and Preissner, K. T. (1996) Eur. J. Biochem. Vol. 241; 557-554). Peptides that contain tandem repeats of GRGDS motifs competitively inhibit the binding of vitronectin to the αVβ3 receptor, which has been shown to promote angiogenesis. In one aspect of the invention, evidence is provided herein that indicates that endostatin inhibits angiogenesis by disrupting the αVβ3-vitronectin interaction.

It is believed that endostatin and compounds exhibiting endostatin-like activity (mimics) may provide effective treatment for cancer. Endostatin may be expensive and difficult to produce. In addition, its effective lifetime in the body may be limited. Therefore, any compounds that exhibit endostatin-like activity and are more easily obtained, or provide for extended pharmacological activity, may be useful in the treatment of cancer, and particularly in those cancers that may respond to angiogenesis inhibitors, such as, for example, metastatic tumors.

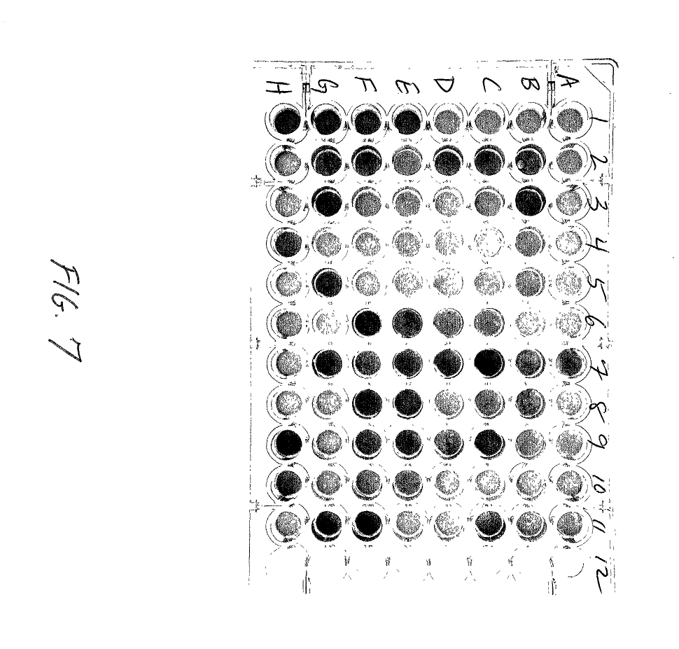

In another aspect of the invention, it is demonstrated that a histidine-tagged synthetic peptide containing two tandem repeats of the GRGDS motif (see Table 1. SEQ. ID 1) binds to dimeric endostatin and that this interaction is competitively inhibited by full length vitronectin, see FIG. 6 .

In yet another aspect of the invention, a high throughput drug screening assay is described that identifies endostatin mimics is described. Histidine-tagged peptides containing two tandem GRGDS motifs are immobilized, from a phosphate buffered saline solution, onto gold nanoparticles (called colloids) that have been derivatized with NTA-Ni-SAMs that capture histidine-tagged species. Endostatin, which is a dimer in its functional form, is added to the colloidal solution. Dimeric endostatin presents two binding sites that recognize GRGDS motif peptides. The binding of dimeric endostatin to two peptides attached to two different colloids, draws the colloids close together and causes the color of the gold colloid solution to change from pink to blue. Recall that colloidal gold appears pink when the particles are dispersed in a homogeneous solution, but the solution turns blue when the particles are aggregated, see FIG. 5. Drug candidates are added to the colloid-immobilized GRGDS motif peptides and endostatin solution. When a drug candidate effectively interrupts the endostatin—GRGDS motif peptide interaction, the solution remains pink. Recall that the drug candidates are monomeric and therefore, even if they bind to the GRGDS peptides, they cannot bring two colloids together, which would cause the solution color to change from the inherent pink, to blue, see FIG. 7.

It should be noted that the high throughput assay identifies compounds that separate into two groups: the first group binds to the GRGDS motif peptide, which mimics full-length vitronectin, and thus inhibits angiogenesis by interrupting the αVβ3-vitronectin interaction. However, the second group of compounds binds to endostatin. Both groups cause the color of the colloidal solution to appear pink, but only compounds that bind to the GRGDS peptide inhibit angiogenesis. Therefore, the high throughput drug screening assay must be followed up with a second assay to determine which of the original compound hits are actual angiogenesis inhibitors. Several assays are available for the secondary screening assay. Compound hits can be tested in a functional matrigel assay to determine which compounds inhibit tubule formation when human umbilical veinous endothelial cells (HUVECs: available from Clonetics) are grown on a matrix of membrane and cell adhesion molecules. Another assay that is used to confirm hits from the particle-based high throughput assay is a typical binding assay performed on peptide-bearing beads, followed by HPLC, to determine which compounds bind to the GRGDS motif peptide (Table 1; SEQ. ID No. 1) and thus function as angiogenesis inhibitors, see Example 5.

```
TABLE 1
Peptide sequences:
SEQ. ID No. 1: GRGDS-containing peptide
            HHHHHHSSSSGSSSSGSSSSGGRGDSGRGDS
SEQ. ID No. 2: FLR peptide
            GTINVHDVETQFNQYKTEAASPYNLTISDVSVSDVPFPFSAQSG
AHHHHHH
```

The invention anticipates that compounds that bind to endostatin may actually enhance angiogenesis and therefore can potentially be used for conditions, such as cardiovascular disease and diabetes, in which it is desirable to enhance vascularization.

The compounds disclosed herein may be administered alone, in combination with each other, and/or in combination with other cancer drugs. It is contemplated that drug therapies may be administered in amounts which are not capable of preventing or reducing angiogenesis when administered alone, but which are capable of preventing or reducing angiogenesis when administered in combination with the compounds disclosed herein. Likewise, is some embodiments, the disclosed compounds may be only effective when used in conjunction with known angiogenesis inhibitors. In some aspects of the invention the effective amount of the compounds disclosed herein is that amount effective to reduce tumor size, prevent tumor growth, prevent new blood vessel growth, prevent the spread of cancer or inhibit metasteses. This can be routinely determined using animal studies. The invasion and metastasis of cancer is a complex process which involves changes in cell adhesion properties which allow a transformed cell to invade and migrate through the extracellular matrix (ECM) and acquire anchorage-independent growth properties. Liotta, L. A., et al., Cell 64:327-336 (1991). Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers. Thus the term “metastasis” as used herein refers to the invasion and migration of tumor cells away from the primary tumor site.

The barrier for the tumor cells may be an artificial barrier in vitro or a natural barrier in vivo. In vitro barriers include e but are not limited to extracellular matrix coated membranes, such as Matrigel. Thus the compounds disclosed herein can be tested for their ability to inhibit tumor cell invasion in a Matrigel invasion assay system as described in detail by Parish, C. R., et al., “A Basement-Membrane Permeability Assay which Correlates with the Metastatic Potential of Tumour Cells,” Int. J. Cancer (1992) 52:378-383. Matrigel is a reconstituted basement membrane containing type IV collagen, laminin, heparan sulfate proteoglycans such as perlecan, which bind to and localize bFGF, vitronectin as well as transforming growth factor-β (TGF-β), urokinase-type plasminogen activator (uPA), tissue plasminogen activator (tPA), and the serpin known as plasminogen activator inhibitor type 1 (PAI-1). Other in vitro and in vivo assays for metastasis have been described in the prior art, see, e.g., U.S. Pat. No. 5,935,850, issued on Aug. 10, 1999, which is incorporated by reference. An in vivo barrier refers to a cellular barrier present in the body of a subject.

A variety of studies involving colloid/colloid interaction can be carried out in accordance with the invention. One set of assays makes use of the effect of an absorptive or emissive species, immobilized with respect to a colloid particle, by a second species that is immobilized with respect to a second colloid particle, brought into proximity or removed from proximity of the first colloid particle by binding, cleavage, or other interaction desirably studied in accordance with the invention. For example, a fluorescent molecule may be immobilized with respect to a first colloid particle and a chemical species having the ability to quench fluorescence of the fluorescent molecule, i.e., effect emission of the fluorescent molecule, can be provided on a second colloid particle. Then, first and second species immobilized with respect to the first and second colloid particles, if they bind to each other, will bring the first and second colloid particles into proximity with each other, causing quenching of the fluorescent molecule. If the first and second species immobilize with respect to the first and second colloid particle, each can bind to a common analyte, then presence of the analyte will cause quenching of fluorescence, and absence of the analyte will avoid quenching. Alternatively, the colloid need not carry an auxiliary signaling element. Intrinsic properties of gold colloids cause the colloids to appear red when dispersed in solution. However, the solution will change color from red to blue when the colloids are forced close together, for example, by a binding interaction. Binding of the two immobilized species on the two sets of colloids may draw the colloids together to result in a change in the solution color from pink to blue.

The invention is useful for treating and/or preventing tumor cell proliferation or metastasis in a subject. The terms “prevent” and “preventing” as used herein refer to inhibiting completely or partially the proliferation or metastasis of a cancer or tumor cell, as well as inhibiting any increase in the proliferation or metastasis of a cancer or tumor cell.

A “subject having a cancer” is a subject that has detectable cancerous cells. Cancers or tumors (malignant and non-malignant) include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas.

A “subject at risk of having a cancer” as used herein is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. When a subject at risk of developing a cancer is treated with one or more of the compounds disclosed herein the subject may be able to kill the cancer cells as they develop or prevent them from spreading.

“Colloids”, as used herein, means nanoparticles, i.e. very small, self-suspendable or fluid-suspendable particles including those made of material that is, e.g., inorganic or organic, polymeric, ceramic, semiconductor, metallic (e.g. gold), non-metallic, crystalline, amorphous, semiconductor nanocrystals, or a combination. Typically, colloid particles used in accordance with the invention are of less than 250 nm cross section in any dimension, more typically less than 100 nm cross section in any dimension, and in most cases are of about 2-30 nm cross section. One class of colloids suitable for use in the invention is 10-30 nm in cross section, and another about 2-10 nm in cross section. As used herein this term includes the definition commonly used in the field of biochemistry.

Certain embodiments of the invention make use of self-assembled monolayers (SAMs) on surfaces, such as surfaces of colloid particles, and articles such as colloid particles having surfaces coated with SAMs. In one set of preferred embodiments, SAMs formed completely of synthetic molecules completely cover a surface or a region of a surface, e.g. completely cover the surface of a colloid particle. “Synthetic molecule”, in this context, means a molecule that is not naturally occurring, rather, one synthesized under the direction of human or human-created or human-directed control. “Completely cover” in this context, means that there is no portion of the surface or region that directly contacts a protein, antibody, or other species that prevents complete, direct coverage with the SAM. I.e. in preferred embodiments the surface or region includes, across its entirety, a SAM consisting completely of non-naturally-occurring molecules (i.e. synthetic molecules). The SAM can be made up completely of SAM-forming species that form close-packed SAMs at surfaces, or these species in combination with molecular wires or other species able to promote electronic communication through the SAM (including defect-promoting species able to participate in a SAM), or other species able to participate in a SAM, and any combination of these. Preferably, all of the species that participate in the SAM include a functionality that binds, optionally covalently, to the surface, such as a thiol which will bind to a gold surface covalently. A self-assembled monolayer on a surface, in accordance with the invention, can be comprised of a mixture of species (e.g. thiol species when gold is the surface) that can present (expose) essentially any chemical or biological functionality. For example, they can include tri-ethylene glycol-terminated species (e.g. tri-ethylene glycol-terminated thiols) to resist non-specific adsorption, and other species (e.g. thiols) terminating in a binding partner of an affinity tag, e.g. terminating in a chelate that can coordinate a metal such as nitrilotriacetic acid which, when in complex with nickel atoms, captures a metal binding tagged-species such as a histidine-tagged binding species. The present invention provides a method for rigorously controlling the concentration of essentially any chemical or biological species presented on a colloid surface or any other surface. Without this rigorous control over peptide density on each colloid particle, co-immobilized peptides would readily aggregate with each other to form micro-hydrophobic-domains that would catalyze colloid-colloid aggregation in the absence of aggregate-forming species present in a sample. This is an advantage of the present invention, over existing colloid agglutination assays. In many embodiments of the invention the self-assembled monolayer is formed on gold colloid particles.

A drug candidate may be studied for competition with the analyte for binding of one of the species, or binding with one site on the analyte. In this case, the analyte may be provided as a known species. Presence of the drug candidate will thus inhibit immobilization of the first and second colloid particles relative to each other, and thus will inhibit quenching. Alternative embodiments involve enhancing emission or shifting the wavelength of emission or absorption of a first molecule, by a second molecule on a second colloid particle.

This colloid/colloid aggregation technique can be used to identify the binding partners of drugs or proteins of interest. This can be accomplished by attaching the drug or protein to one set of colloids and possible binding partners to other sets of colloids and assaying for a binding interaction between the two sets of colloids. Once a biological target of a drug or protein has been identified, candidate drugs can be added to the assay in the presence of the colloid-attached binding partners to disrupt binding of the drug or protein to the cognate ligand, allowing identification of synthetic mimics of the drug or protein on the first set of colloids. This technique is very useful in identifying the biological target of orphan drugs or uncharacterized proteins for diagnostic or drug-screening purposes. This technique will also allow identification of synthetic replacements or “mimics” of currently used drugs that are expensive or difficult to produce.

In one embodiment, an angiogenesis inhibitor is attached to one set of colloids (via an affinity tag linkage, chemical coupling, or nonspecific adsorption), and its biological target is attached to another set of colloids. For the unique case of an angiogenesis inhibitor that has two or more ligand-binding sites, such as endostatin, the ligand may be attached to one set of colloids and the angiogenesis inhibitor may be added in solution. Drug candidates are added and assayed for their ability to disrupt the binding interaction. Any drug that inhibits the interaction is then attached to a third set of colloids and assayed for binding to the angiogenesis inhibitor and the biological target of the angiogenesis inhibitor. A drug that binds to the biological target of the angiogenesis inhibitor and inhibits binding of the angiogenesis inhibitor to its target can be deemed a “mimic” of the angiogenesis inhibitor, and may be used as a replacement drug. This assay may be used to screen for mimics of virtually any drug. It is of specific interest for drug screening for synthetic replacements of angiogenesis inhibitors, which are both costly and difficult to produce. The assay can be used to identify synthetic replacements for endostatin, through disruption of the endostatin-vitronectin or endostatin-RGD-peptide interactions; angiostatin, through disruption of the angiostatin-ATP-synthase or angiostatin-vitronectin interaction; or TNP-470 through disruption of the TNP-470-methionine-aminopeptidase interaction. As in other colloid/colloid assays, color change, fluorescence quenching, or other emissive molecule enhancement or suppression and the like can be indications of a result. Study of RGD/endostatin interaction is described in examples 1 and 2 below.

This colloid/colloid aggregation technique also can be used for discovery of angiogenesis inhibitors or ligands involved in angiogenesis pathways. In one assay, suspected angiogenesis inhibitors or proteins can be immobilized relative to (e.g., fastened to) a first colloid particle. Second colloid particles can be immobilized with respect to molecules that have been implicated in angiogenesis and/or metastasis, such as basement membrane proteins, integrins, or adhesion molecules. If a particular angiogenesis inhibitor binds to the basement membrane protein, integrin, or adhesion molecule immobilized on the second set of colloids, then the two sets of colloids will become immobilized with respect to each other and the binding interaction will become detectable by methods of the invention such as color change, precipitation, etc. Once an angiogenesis inhibitor is identified by this method, candidate drugs for disruption of the binding can be screened. If the drugs disrupt interactions, then colloid particles will not immobilize relative to each other or will do so to a lesser degree. This assay can be used with known angiogenesis inhibitors to identify or verify the biological targets of the angiogenesis inhibitors. Drug candidates can then be added to the assay to identify other drugs that act on the same biological target.

Another embodiment in which colloid particles can be immobilized relative to each other in such assays involves colloids each being immobilized with respect to a common surface. The common surface can be a surface of another colloid particle presenting binding partners of species on the first colloid particles. The common surface can also be the surface of an article such as a membrane such as a nitrocellulose membrane, a chip surface, a surface of an article derivatized with a SAM, or the like. In preferred embodiments, the surface to which the colloid particles can bind includes binding sites at a high enough density so that if binding occurs (between species on the common surface and species on the colloid particles), the colloid particles will be brought into close enough proximity that detection (via color change characteristic of aggregation, quenching of fluorescence, or other property described herein) can occur.

When administered, the formulations of the invention are applied in pharmaceutically acceptable amounts and in pharmaceutically acceptable compositions. Such preparations may routinely contain salts, buffering agents, preservatives, compatible carriers, and optionally other therapeutic ingredients. Such pharmacologically and pharmaceutically acceptable salts include, but are not limited to, those prepared from the following acids: hydrochloric, hydrobromic, sulfuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts of the carboxylic acid group.

According to the methods of the invention, the composition may be administered in a pharmaceutically acceptable carrier. In general, pharmaceutically-acceptable carriers are well-known to those of ordinary skill in the art. As used herein, a pharmaceutically-acceptable carrier means a non-toxic material that does not interfere with the effectiveness of the biological activity of the active ingredients. Pharmaceutically acceptable carriers include diluents, fillers, salts, buffers, stabilizers, solubilizers and other materials which are well-known in the art. The compositions of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces locally administering the compositions of the invention, including as implants.

In administering the compounds of the invention to subjects, dosing amounts, dosing schedules, routes of administration and the like may be selected so as to affect the other known activities of these compounds. For example, amounts, dosing schedules and routes of administration can be selected as described herein, whereby therapeutically effective levels for angiogenesis inhibition are provided, yet therapeutically effective levels for alternative treatments are not provided.

According to the methods of the invention, the compositions can be administered by injection by gradual infusion over time or by any other medically acceptable mode. The administration may, for example, be intravenous, intraperitoneal, intramuscular, intracavity, subcutaneous or transdermal. Such modes of administration as those described above as well as oral, rectal, topical, nasal, transdermal or parenteral routes may be used. Preparations for parenteral administration includes sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents, and inert gases and the like. Those of skill in the art can readily determine the various parameters for preparing these alternative pharmaceutical compositions without resort to undue experimentation.

Compositions of the invention are given in dosages, generally, given at the maximum amount while avoiding detrimental side effects.

One of skill in the art can determine what an effective amount of a composition is by screening the ability of the composition to any of the assays described herein. Effective amounts will depend, of course, on the severity of the condition being treated; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

Another aspect of the present invention involves a method comprising providing any of the structures as disclosed herein or as determined from any of the assays described herein and performing a combinatorial synthesis on any one of those structures, preferably to obtain a derivative of the composition. For example, the effectiveness of a composition may be enhanced if it has greater polarity. Thus, the composition is reacted with a variety of electron donating or withdrawing groups in a combinatorial fashion to obtain a composition (i.e. derivative) of greater polarity. An assay is performed with the derivative to determine its effectiveness in angiogenesis inhibition. The combinatorial synthesis can involve subjecting a plurality of the compositions described herein to combinatorial synthesis.

The compositions may conveniently be presented in unit dosage form and may be prepared by any of the methods well known in the art of pharmacy. In general, the compositions are prepared by uniformly and intimately bringing the active compounds into association with a liquid carrier, a finely divided solid carrier, or both, and then, if necessary, shaping the product.

Compositions suitable for oral administration may be presented as discrete units such as capsules, cachettes, tablets, or lozenges, each containing a predetermined amount of the active compound. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. Nos. 4,748,034 and 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Use of a long-term sustained release implant may be particularly suitable in some cases. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above.

### EXAMPLE 1

### Detection of the Biotin—Streptavidin Interaction Using Gold Colloids

Gold colloids were prepared using a mixture of 10 μM biotin thiol, 10 μM NTA thiol, and 580 μM C11 thiol. Control colloids were prepared using 20 μM NTA thiol and 580 μM C11 thiol for a total thiol concentration of 600 μM. After deposition, the colloids were heat cycled in 400 μM EG3 thiol, and charged with nickel sulfate. A streptavidin stock solution (1 mg/mL) was prepared in 10 mM sodium phosphate buffer, 100 mM sodium chloride, pH 7.4 (buffer) To detect the biotin-streptavidin interaction, 60 μL buffer, 10 μL streptavidin, and 30 μL colloids, were mixed in a well of a 96-well plate. The plate was incubated at room temperature and observed for color change. At the three highest concentrations of streptavidin (0.1 mg/mL, 0.01 mg/mL, 0.001 mg/mL) the colloids presenting biotin turned blue (wells A1, A2, A3, FIG. 5). At lower concentrations of streptavidin, the wells containing biotin colloids remained pink (wells A4, A5, A6, A7). A similar experiment was performed using colloids presenting only the NTA group (wells B1-B7). All of these wells remained pink, demonstrating that the color change is specific for the biotin-streptavidin interaction.

FIG. 5 is a digital photo of a colorimetric nanoparticle experiment in which gold colloids were derivatized with either biotin-SAMs (top row) or NTA-Ni-SAMs (bottom row). Streptavidin which has four binding sites for biotin, and thus will cross-link biotin-presenting colloids, was added free in solution. The concentration of the streptavidin that was added decreases from left to right. As can be seen, wells containing sufficient amounts of streptavidin, cross-linked the biotin-presenting colloids and caused the solution to turn blue. The same concentrations of streptavidin were added to the bottom row that held the control colloids that presented NTA-Ni, however no binding occurred and the solution color remained pink.

### EXAMPLE 2

### The Angiogenesis Inhibitor, Endostatin Specifically Binds to a His-Tagged GRGDS Motif Peptide (HHHHHHSSSSGSSSSGSSSSGGRGDSGRGDS), but Angiostatin does not

200 μL NTA-Ni agarose (Qiagen) were washed 2× with 100 μL ddH20, then with wash buffer A, containing 50 mM NaH2PO4, 300 mM NaCl, and 10 mM imidazole at pH 8.0.

A synthetic peptide, was dissolved in DMSO then diluted in phosphate buffer to a final concentration of 1 mM. 100 μL of this peptide solution was incubated with the NTA-Ni resin for 20 minutes at room temperature to allow binding of the histidine-tagged peptide to the NTA-Ni resin. The resin was then pelleted and the supernatant was removed. The resin was washed in buffer A. The peptide bound resin was then divided into two aliquots. A first aliquot was mixed with 100 μL human recombinant endostatin (0.1 mg/mL in 10 mM sodium phosphate buffer, 100 mM sodium chloride, pH 7.4, diluted from stock endostatin, Calbiochem 324746). A second aliquot was mixed with 100 μL human angiostatin, Calbiochem 176700(0.1 mg/mL in 10 mM sodium phosphate buffer, 100 mM sodium chloride, pH 7.4. The beads and angiogenesis inhibitors were incubated on ice 15-20 minutes to allow binding to the bead-immobilized peptide. The resin was then pelleted. The supernatants were removed and reserved for analysis by SDS PAGE (flow through). The beads were then washed 2× with 10 mM sodium phosphate buffer. The histidine-tagged peptides and any immobilized drug were eluted by the addition of 4 aliquots of an imidazole (250 mM) wash.

Analysis of the eluate and flow through by SDS PAGE showed that endostatin co-eluted with the GRGDS motif peptide but angiostatin did not.

### EXAMPLE 3

### Drug Screen for Synthetic Mimics of Endostatin

40 μM NTA colloids presenting a His-tagged peptide containing a tandem repeat GRGDS motif (Sequence ID No.1; Table 1) were prepared by incubating 2.1 mL colloids with 210 μl 100 μM His-RGD for ten minutes pelleting the colloids to remove excess unbound peptide, and resuspending the colloids in 10 mM sodium phosphate buffer (pH 7.4). Negative control colloids were prepared by substituting an irrelevant His-tagged FLR peptide (Sequence ID No. 2, see Table 1). 25 μl GRGDS-colloids (or random peptide-colloid for negative controls) was added to each well of a 96-well plate along with 65 μl sodium phosphate buffer per well. DMSO was added in place of a drug to the positive and negative controls. 5 μl of 0.1 mg/ml endostatin (Calbiochem) was added to each well. The plate was incubated in room temperature and observed for color change. After about 20 minutes, the positive controls changed color from pink to blue as the endostatin bound to the GRGDS peptide. Negative control wells remained pink, since endostatin did not bind to the random peptide. A color change from pink to blue in the wells containing drug candidates indicates that the drug did not effect binding of endostatin to GRGDS. A lack of color change from pink to blue (the well remains pink over time) indicates that the drug candidate bound to either the GRGDS or the endostatin and disrupted the binding interaction between endostatin and GRGDS peptide. Drug identified in this manner that bound to the GRGDS motif and inhibited binding of endostatin could be used as synthetic replacement of endostatin.

FIG. 7 is a digital photo of a drug screening plate in which drug candidates were separately tested in wells of a multi-well plate for their ability to interrupt the endostatin-GRGDS-containing peptide interaction. The pink color of well C9 indicates that it contains a drug that mimics endostatin.

### EXAMPLE 4

### In vitro Matrigel Assay for Testing Angiogenesis Inhibitors

Matrigel (Becton Dickinson 354234) was thawed on ice until use. 200 ul Matrigel was added to each well of a 24-well tissue culture plate and incubated at 37° C. for at least 30 minutes to allow solidification. HUVEC cells grown in a T-75 flask were removed by incubating with a trypsin-EDTA solution for about 8 minutes. After detachment, 30 ml of EGM-2 media (Clonetics, CC-4176) was added to inactivate the trypsin. The cells were pelleted by centrifugation, and resuspended in 2 ml EGM-2. An aliquot was counted in a hemacytometer and the cell solution adjusted with media to a final concentration of 5×105 cells/ml. Each drug was tested by mixing 0.5 ul (from the DMSO stock) with 50,000 HUVEC cells (100 ul). This mixture was added to each Matrigel-coated well. After 24 hours, each well was observed by light microscopy for the presence or absence of tubule-like structures. Drugs which prevented the formation of these tubule structures were scored as angiogenesis inhibitors.

### EXAMPLE 5

### Detection of Drug Binding to the FLR Peptide by HPLC

1.8 ml Ni-NTA agarose (Qiagen) was first washed with 20 ml sodium phosphate buffer (10 mM sodium phosphate, 100 mM sodium chloride, pH 7.4). After washing, the resin was resuspended in 45 ml of sodium phosphate buffer containing 9 mg of the FLR peptide (sequence ID #2). The mixture was incubated at room temperature for 30 minutes to allow binding of the His-tagged FLR peptide to the NTA resin. After binding, the resin was pelleted by centrifugation, and washed twice with 10 ml phosphate buffer. After the second wash, the resin was resuspended in 9 ml sodium phosphate buffer. Nine microcentrifuge tubes were prepared which contained different drugs (20 uL of a mg /mL DMSO stock dissolved in 900 uL sodium phosphate buffer). 1 ml of resin presenting the FLR peptide was added to each tube and incubated at room temperature for 30 minutes to allow binding of the drug. After binding, the resin was pelleted, and washed twice with 1.0 ml sodium phosphate buffer. The FLR peptide was eluted with 100 ul of 250 mM imidazole in sodium phosphate buffer. The eluate was analyzed by HPLC using a C-18 reverse phase column. If the drug bound to the FLR peptide, two major peaks were observed. If no drug was bound, only a peak corresponding to the peptide was observed. The elution times were compared to control samples containing each drug alone, or the FLR peptide. To demonstrate the specificity of the drug—FLR interaction, a control experiment was performed using an irrelevant RGD peptide (Sequence ID No. 1).

### EXAMPLE 6

### Vitronectin Inhibits Binding of Endostatin to the GRGDS Peptide

40 μM NTA gold colloids were prepared which presented the His-tagged RGD peptide (Sequence ID No.1, see Table 1). These colloids were mixed with endostatin (0.1 mg /mL) and turned blue, indicating binding of endostatin to the GRGDS peptide (A1, A2, FIG. 6). Control colloids presenting an irrelevant FLR-peptide (Sequence ID #2, see table 1) remained pink (wells A3, A4). At the highest concentration of vitronectin (0.1 mg /ml), the endostatin—GRGDS interaction is disrupted, and the well remains pink (B1). At lower concentrations of vitronectin, the endostatin—GRDS interaction is not affected and the wells turn blue (B2-B5).

FIG. 6 is a digital photo of a colorimetric nanoparticle experiment that shows that the GRGDS-containing peptide (SEQ. ID No. 1) interacts with dimeric endostatin, wells A1 and 2 and that this interaction is competitively inhibited by the addition of full-length vitronectin, well B1.

### EXAMPLE 7

### Determination of Colloid/Colloid Linkage via Protein/Protein Recognition on NTA-Presenting Colloids

600 microliters colloids, derivatized with a self-assembled monolayer that presents nitrilo tri-acetic acid, NTA (for the capture of histidine-tagged proteins), was mixed with 60 microliters of 500 micromolar histidine-tagged RGD-motif-containing peptide. 600 microliters of a second set of NTA-Ni presenting colloids was mixed with 60 ul of a 500 uM solution of histidine-tagged GST, an irrelevant protein, as a negative control. Colloids were spun down and resuspended in phosphate buffer to remove residual unbound protein or peptide. Endostatin and Angiostatin, two proteins implicated in angiogenesis and suspected of binding to regions of vitronectin, such as the RGD peptide, were prepared by dialysis into phosphate-buffered saline solution and then 1:10 dilution into PBS from a stock concentration of 1 mg/mL.

400 ul phosphate buffer (pH 7.4), 200 ul either RGD-bound colloids or GST-bound colloids, and either 100 ul endostatin or angiostatin, 50 ul endostatin or angiostatin/50 ul phosphate buffer, or 100 ul phosphate buffer (as a negative control) were added to each well of a crystallization dish. Color change was monitored over time at room temperature. After approximately 15 minutes, a color change from red to blue was visible in wells that contained RGD-peptide-bound colloids and endostatin. The color change was more pronounced in the well that contained 100 ul endostatin than in the well that contained 50 ul endostatin/50 ul phosphate buffer. No color change occurred in the wells containing endostatin and GST-bound colloids or angiostatin and RGD- or GST-bound colloids. The results show that endostatin binds to RGD-motif-containing peptides, and that endostatin is able to bind to two or more RGD-peptides, thus linking the colloids, and causing a color change from red to blue. The results were verified by gel electrophoresis. The RGD peptide and histidine-tagged GST were bound to a small amount of NTA-Ni agarose resin at saturating concentration. Endostatin was incubated with the resin and allowed to bind to the protein on the resin. The histidine-tagged proteins were then eluted using imidazole, and the samples were analyzed by SDS-PAGE. The results clearly showed that endostatin bound to the resin-immobilized RGD peptide and eluted with the protein off of the resin, while it did not bind to the resin-immobilized GST.

This assay could be easily adapted for screening of drug candidates that either mimic the RGD-binding characteristic of endostatin or bind to the RGD-binding domains on endostatin. Drug candidates could be added to the RGD-colloids in the presence of endostatin to look for drugs that inhibit the color change from red to blue.

### EXAMPLE 8

### Identification of Endostatin Mimics via RGD/Endostatin Interaction

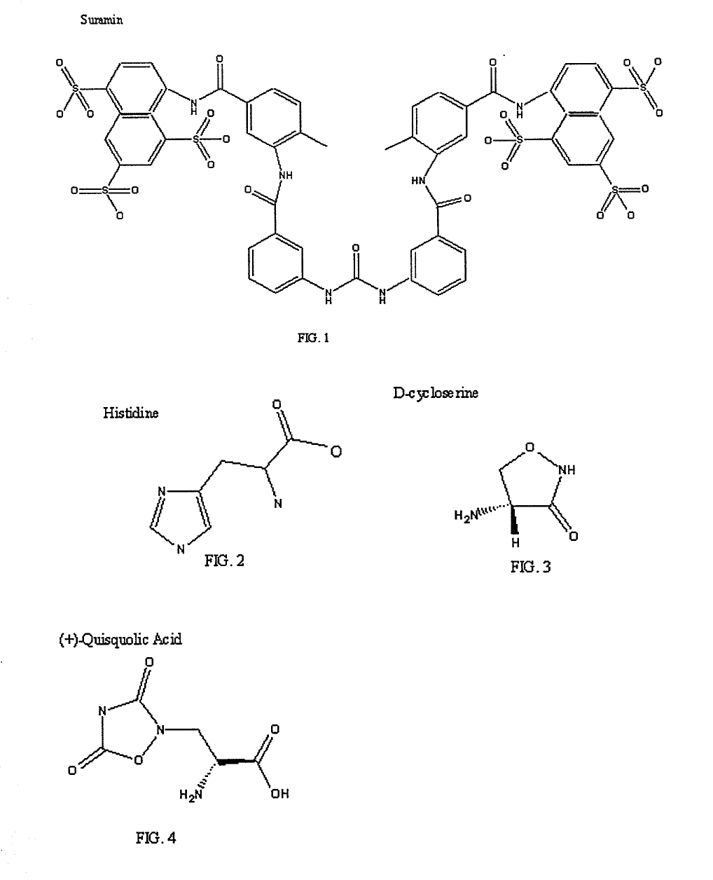

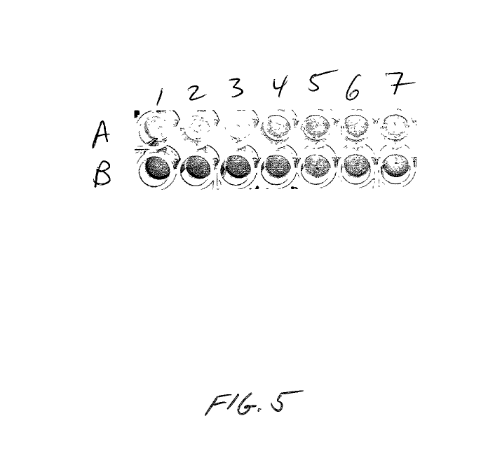

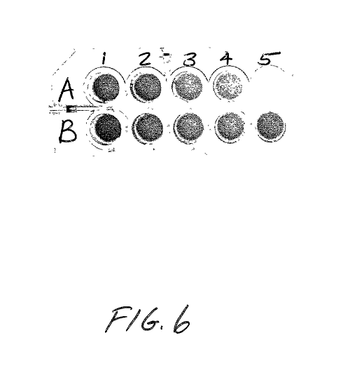

60 uM NTA colloids presenting a His-tagged peptide containing cyclic RGD motifs (GRGDSGRGDS) were prepared by incubating 1 mL of colloids with 200 ul 100 uM His-RGD for ten minutes, pelleting the colloids to remove excess unbound peptide, and resuspending the colloids in PBS. Negative control colloids were prepared by substituting a random His-tagged peptide in place of the RGD peptide. 30 μL RGD-colloids (or random peptide-colloids for negative controls) were added to each well of a 96-well plate along with 60 ul PBS per well. 5 μL of a candidate drug at 2.9 mg/ml in DMSO was added to each well of the plate. Candidate drugs including L-histidine, D-cycloserine, quisqualic acid and suramin were assessed. (See FIGS. 1-4). DMSO was added in place of a drug to each of the positive and negative controls. 4.75 ul of 0.1 mg/mL endostatin was added to each well. The plate was incubated at room temperature and observed for color change. After about 20 minutes, the positive controls changed color from pink to blue as the endostatin bound to the RGD peptide. Negative control wells remained pink, since endostatin did not bind to the random peptide. A color change from pink to blue in the wells containing drug candidates indicated that the drug did not affect binding of endostatin to RGD. A lack of color change from pink to blue (the well remains pink) indicated that the drug candidate bound to either the RGD or the endostatin and disrupted the binding interaction. Drugs identified in this manner that bound to the RGD motif and inhibited binding of endostatin may be endostatin mimics and could be used as synthetic replacements of endostatin. Using this assay, four compounds were identified that disrupted the RGD-endostatin interaction: L-histidine, quisqualic acid, suramin, and D-cycloserine.

The above description and examples are meant to be taken as exemplary only, of preferred embodiments of the invention. As such, the invention can be practiced according to other techniques and equivalents thereof.

What is claimed is:

1. A method comprising treating a human patient susceptible to or exhibiting symptoms of invasive cancer, by administering to the patient a therapeutically effective amount of a composition selected from the group consisting of L-histidine, Quisqualic Acid, D-cycloserine, Suramin and analogs of any of these, the administering of the therapeutically effective amount of the composition not being otherwise indicated for the patient.

2. A method comprising treating a human patient susceptible to or exhibiting symptoms of metastatic tumors, by administering to the patient a therapeutically effective amount of a composition selected from the group consisting of L-histidine, Quisqualic Acid, D-cycloserine, Suramin and analogs of any of these, the administering of the therapeutically effective amount of the composition not being otherwise indicated for the patient.

3. A method comprising treating a human patient where angiogenesis inhibition is indicated, by administering to the patient a therapeutically effective amount of a composition selected from the group consisting of L-histidine, Quisqualic Acid, D-cycloserine, Suramin and analogs of any of these, the administering of the therapeutically effective amount of the composition not being otherwise indicated for the patient.

4. A method comprising treating a human patient wherein treatment with endostatin has been indicated, by administering to the patient a therapeutically effective amount of a composition selected from the group consisting of L-histidine, Quisqualic Acid, D-cycloserine, Suramin and analogs of any of these.
